(19) United States
(12) Patent Application Publication
(10) Pub. No.: US 2014/0257449 A1
(43) Pub. Date: Sep. 11, 2014

(54) CONDUCTIVE GARMENT

(71) Applicant: Jace Helmer, Carlsbad, CA (US)

(72) Inventor: Jace Helmer, Carlsbad, CA (US)

(21) Appl. No.: 14/195,691

(22) Filed: Mar. 3, 2014

Related U.S. Application Data
(60) Provisional application No. 61/773,703, filed on Mar. 6, 2013.

(51) Int. Cl. A61N 1/04

(52) U.S. Cl. CPC A61N 1/0484
USPC 607/148

(57) ABSTRACT

A conductive garment. Conductive fabric sections are each separated by a nonconductive strip. An electrical contact is connected to each conductive fabric section. An attachment pad connects the conductive garment to a support wrap. The nonconductive strip is a sufficient width so that it is able to cover a section of the human body where the user does not want electricity applied. In a preferred embodiment, the nonconductive strip is of sufficient width to cover the spine of a human being.

The present invention relates generally to an electrical stimulator, and in particular, to therapeutic conductive garment systems used to provide pulsed electrical stimulation to nerve, muscle, skin, circulatory and/or other tissue of a human or animal subject. This application claims the benefit of U.S. Provisional Application Ser. No. 61/773,703, filed on Mar. 6, 2013, which is incorporated by reference herein.

### BACKGROUND OF THE INVENTION

Conductive garments providing electrical stimulation have been found to provide therapeutic benefit to various biological tissue. For example, transcutaneous electrical nerve stimulation (TENS) has been used to treat neuromuscular injuries and other conditions by stimulation of muscle and nerve tissue. Electrical stimulation has also been found to increase circulation, promoting faster healing of injuries and preventing adverse conditions resulting from poor circulation.

Electronic Muscle Stimulator (EMS) uses a cycle of stimulation, contraction and then relaxation at intensities from 1 to 130 Mh to increase blood flow to the area, which decreases inflammation and promotes healing and muscle growth. By stimulating the muscles at their basic structure, an EMS unit causes muscle contractions similar to those obtained by exercise. The EMS units are specifically used to prevent atrophied muscles or for retraining muscles, such as in an individual who has been paralyzed or partially paralyzed. In addition, EMS is used for many other applications such as range of motion improvement, increased circulatory action and the prevention of blood clots.

Electrical stimulation may be delivered to treated tissue by one or more electrodes, such as adhesive patch electrodes or garment electrodes. Conductive garments are preferred for patients who are skin-sensitive to conventional gelled electrodes.

FIGS. 1, 2 and 2B show prior art conductive garment 1. Velcro® hook section 2 is utilized to securely adhere conductive garment to the inside surface of a conductive support wrap. Conductive fabric 4 is a highly conductive silver fiber material that offers low impedance and comfortable stimulation. Conductive garment 1 includes foam pad 5 to retain water or conductive Electro-Mist spray on the conductive surface. Conductive garment 1 includes snap connector 6 with pin adapter to accept snap or male 2 mm pin from lead wire.

Prior art conductive garment 1 may be utilized as described. However, there are some areas of the human body that should not be subject to electrical stimulation, such as the spine. When utilizing prior art conductive garment 1, the user must avoid the spine. Therefore it often becomes necessary to utilize multiple conductive garments.

What is needed is an improved conductive garment.

### SUMMARY OF THE INVENTION

The present invention provides a conductive garment. Conductive fabric sections are each separated by a nonconductive strip. An electrical contact is connected to each conductive fabric section. An attachment pad connects the conductive garment to a support wrap. The nonconductive strip is a sufficient width so that it is able to cover a section of the human body where the user does not want electricity applied. In a preferred embodiment, the nonconductive strip is of sufficient width to cover the spine of a human being.

### BRIEF DESCRIPTION OF THE DRAWINGS

FIGS. 1, 2 and 2B show a prior art conductive garment.

FIGS. 3 and 4 show preferred embodiments of the present invention.

FIG. 5 shows another preferred embodiment of the present invention.

FIG. 6 shows the utilization of a preferred embodiment of the present invention.

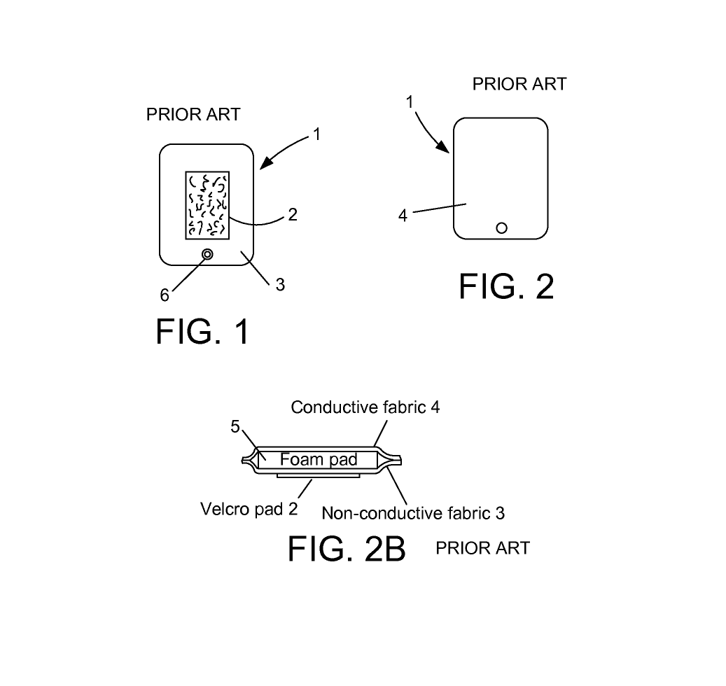

### DETAILED DESCRIPTION OF THE PREFERRED EMBODIMENTS

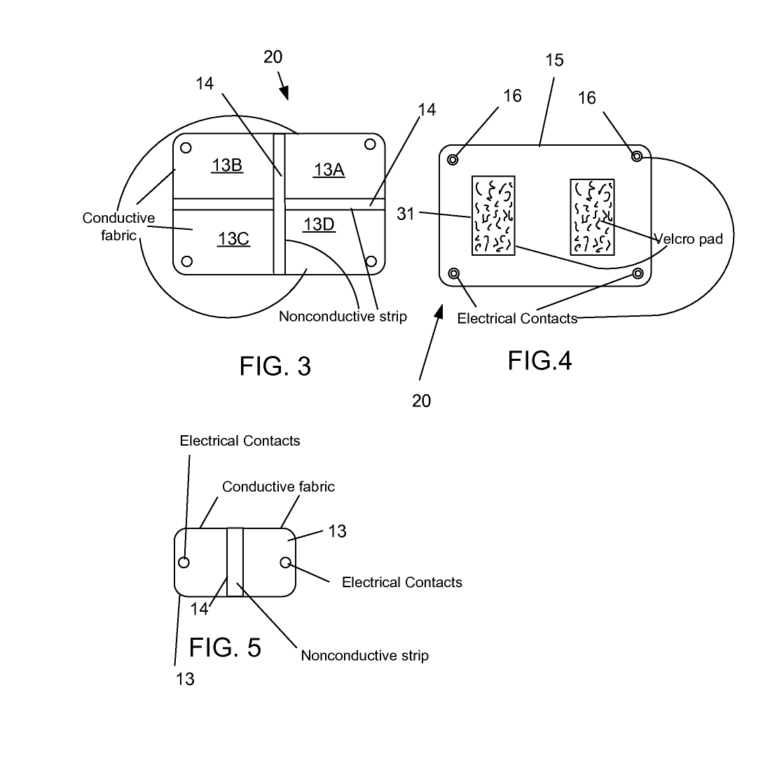

FIGS. 3 and 4 show a preferred embodiment of the present invention. Conductive garment 20 includes nonconductive strip 14 separating conductive fabric sections 13A-13D. Each conductive fabric section 13A, 13B, 13C and 13D includes its own snap connector 6 to accept an electrical wire input from an electrical source. Each conductive garment section 13A-13D can then be electrically stimulated as desired by the user. Additionally, the user may place nonconductive strips 14 over a part of the body that should not be receiving electrical stimulation, such as the spine. The spine will then not be subjected to electrical stimulation. For example, the utilization of conductive garment 20 allows the patient to only apply one conductive garment, with nonconductive strip 14 covering the spine.

FIG. 5 shows another preferred embodiment of the present invention. Nonconductive strip 14 separates conductive fabric sections 13 as shown. Each conductive fabric section 13 may then be stimulated electrically as described above.

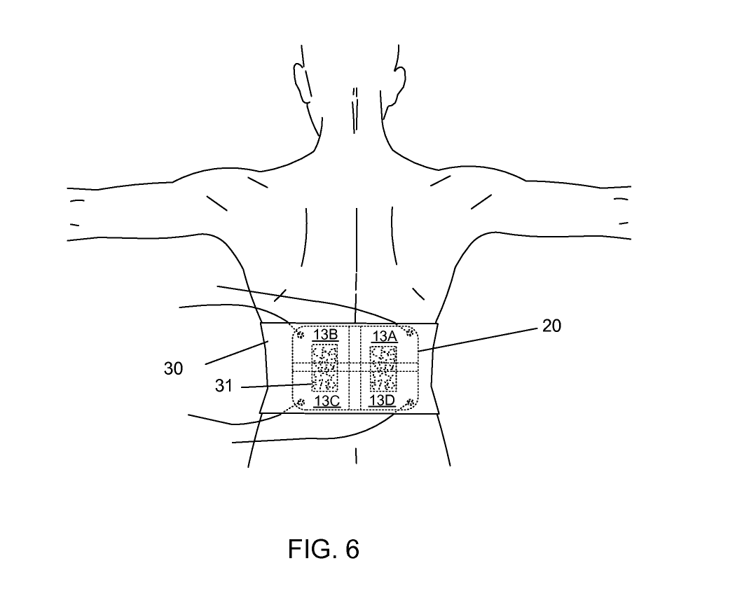

FIG. 6 illustrates a preferred utilization of conductive garment 20. Conductive garment 20 is being held in place by support wrap 30. Velcro® hook pads 31 connect conductive garment 20 to support wrap 30. Conductive garment 20 is being utilized to provide electrical stimulation to the user's lower back. The user's spine is covered by conductive garment 20. However, nonconductive strip 14 is directly over the spine so that conductive fabric sections 13A-13D are not in contact with the spine. In a preferred embodiment, nonconductive strip 14 is a least one inch wide. Electrical signals can be applied to electrical contact snap connectors 16 so that conductive fabric sections 13A-13D electrically stimulate the patient as desired. It should be noted that each conductive fabric section 13A-13D can be provided with its own specific electrical signal independent of the other sections. This can provide the user with a customized treatment.

FIG. 6 shows nonconductive strip 14 of conductive garment 20 utilized to cover the user's spine. It is also possible to utilize conductive garment 20 to cover other areas of the user's body, such as the hand, forearm, foot or calf. Nonconductive strip 14 would be utilized to cover other sections of the body where the user does not want electricity applied.

In a preferred embodiment conductive fabric section 13 is fabricated from a weave that includes: 80 percent polyester, 10 percent nylon and 10 percent nylon with silver complex. The nonconductive strip is preferably nylon.

Although the above-preferred embodiments have been described with specificity, persons skilled in this art will recognize that many changes to the specific embodiments disclosed above could be made without departing from the spirit of the invention. Therefore, the attached claims and their legal equivalents should determine the scope of the invention.

What is claimed is:

1. A conductive garment, comprising:
A. a plurality of conductive fabric sections,
B. at least one nonconductive strip separating each of said plurality of conductive fabric sections,
C. an electrical contact connected to each of said plurality of conductive fabric sections,
D. a support wrap,
E. an attachment pad for connecting said conductive garment to said support wrap
wherein said nonconductive strip is of sufficient width to cover a section of the body where the user does not want electricity applied.

2. The conductive garment of claim 1, wherein said plurality of conductive garments is two conductive garments and said at least one nonconductive strip is one nonconductive strip.

3. The conductive garment of claim 1, wherein said plurality of conductive garments is four conductive garments and said at least one nonconductive strip is two nonconductive strips.

4. The conductive garment as in claim 1, wherein said attachment pad is a Velcro® hook attachment pad.

5. The conductive garment as in claim 1 wherein said width is at least one inch.

6. The conductive garment as in claim 1 wherein said at least one nonconductive strip is placed over a human spine.

7. The conductive garment as in claim 1, wherein said plurality of conductive fabric sections include a weave comprising:
A. 80 percent polyester,
B. 10 percent nylon, and
C. 10 percent nylon with silver complex.

8. The conductive garment as in claim 1, wherein said nonconductive strip is nylon.

9. The conductive garment as in claim 1, wherein said section of the body where the user does not want electricity applied is the spine.

10. The conductive garment as in claim 9, wherein width is one inch.
